SEQUENCE LISTING (14 sequences)

SEQ ID NO: 1 - DNA, 1736 bp, Exiguobacterium sibiricum
  CDS (101)..(1633)
  sig_peptide (101)..(166)
  mat_peptide (683)..(1633)
tactatattcagaaaattctaattaatacgaacggcttattctcgtttcatcctttctttggcatagttagactcaacccaaacaaggaggcgatttattttgaaaaagtttctcgctacatcgcttgtcgcaagtgtacttgtcgttcctacagtcgtcggagcagaaggtcttcaagccggtaaactgacgaaagcatcatcagaaccagctgcatcaatcgttaaagagtacgtcaacaaaaaaggtgatttctctgtccaagatgtccaaaaagatggatcatctaacatcgtacgtctccaacaagaagtggatggcgtcccggtcttcggtagtgtcgtcgtcggtaacgttgcgaaagacggtactttgaaagcagtcgtaaatgatgcaatcaacgtcaaagggaaacctggtctcgctaaaaaagcatcactctctgagaaaaaagcacttaaactttatcaaaaagccatcaaagcaacagaattcgaagtggctcctaaagcagaactcgtcatctatccagtcaaagacgacgcggtctatgcttacaaagtcacatcaactgtcctcgccggcaaagagccgtcacgctggacttacttcatcgatgcgaattcaggtaaagttcttaataaatacgaccaactcgctcacgcaaaaccggtcaacacagttaccggaacgacttctgtcggtacaggtactacggttctcggaactactgccactttcaacacagtgaagagcggttcttactactacttacaagattcgacacgcggtaaagggatctacacgtacgatgcaaaaaaacgtaacacgcttccaggttcgctttgggcggatcttgataaccaattcaatacaacatacgaccgtgccgctgtcagcgcgcaagttaatgcagtaaaaacgtatgatttctacaaaaacacatacggccgcaacagttatgacaacgcaggtgctgcacttaactcttcagtccactattcaacgagctacaacaatgcgttttgggatggaacgaagatggtctatggtgatggagacggttcaacattcacatacctctctggcgcacttgacgttgttgcccacgaattgacgcatgccgtcacagaatacacagctggactcgtctatcaaaatgagtcgggtgccatcaacgaagcggtttccgatatcatgggaacagttgctgaatactcagtcggatcgaacttcgattggcttgtaggagaagacatctatacacctggtgtcagcggcgacgcactccgttcgatgtctaacccggctgcttacggcgatccggaccactactcaaaacgctacacaggtacacaggataacggtggtgtccacacgaactccggtatcgtcaacaaagccgcttaccttctcggtaacggtggaacacacactggtgtcacagtcactggtgtcggcgtaccgaaactcggcgcaatctactaccgtgccctcagcgtttacttgactccgaactcgaacttcagctcgctccgcgcagcagtcgttcaatcagcgaaagacctttacggttcgacaagtgcagaagcaacagcagcagcgaaatcatttgatgctgtcggcgtctactaagaaaacttcctcccaaggattttcgacctcctgagtccatgactcgggaggttttttttacgaaaaaaaacgatctgtcaaaaacagaccgttccggttt

SEQ ID NO: 2 - protein, 511 aa, Exiguobacterium sibiricum
MKKFLATSLVASVLVVPTVVGAEGLQAGKLTKASSEPAASIVKEYVNKKGDFSVQDVQKDGSSNIVRLQQEVDGVPVFGSVVVGNVAKDGTLKAVVNDAINVKGKPGLAKKASLSEKKALKLYQKAIKATEFEVAPKAELVIYPVKDDAVYAYKVTSTVLAGKEPSRWTYFIDANSGKVLNKYDQLAHAKPVNTVTGTTSVGTGTTVLGTTATFNTVKSGSYYYLQDSTRGKGIYTYDAKKRNTLPGSLWADLDNQFNTTYDRAAVSAQVNAVKTYDFYKNTYGRNSYDNAGAALNSSVHYSTSYNNAFWDGTKMVYGDGDGSTFTYLSGALDVVAHELTHAVTEYTAGLVYQNESGAINEAVSDIMGTVAEYSVGSNFDWLVGEDIYTPGVSGDALRSMSNPAAYGDPDHYSKRYTGTQDNGGVHTNSGIVNKAAYLLGNGGTHTGVTVTGVGVPKLGAIYYRALSVYLTPNSNFSSLRAAVVQSAKDLYGSTSAEATAAAKSFDAVGVY

SEQ ID NO: 3 - DNA, 1730 bp, Exiguobacterium AT1b
  CDS (101)..(1627)
  sig_peptide (101)..(166)
  mat_peptide (683)..(1627)
aaatactcaatattatgaattatcaaaataattaaaaagatggatacagcctttgtgggaaagtgtgtaaagtaagaatcatcacaaaggaggtaaacacatgaaaaaagttgtttctacatcattaatcgcaggcgtcttgctcgtcccacaattggtcggtgcggcagaattgaagtcaggcactctgacgaaaccatcagagagtgcgccgacaacgattgtaaaagagtacgtcaaatcaaaaggtgagttcaagacgatcgagtcgaagtcagacaaagtcgggaaagtggttaaactccaacagacagttgatggcgtgccagtattcggtggtgtggtagtcggtgtcgtagatgaagcgggtcaattgaagacagtcgtggatgatgcgaaatcggtcaagaacctacataagtcgatcaagttaactgaaaagaaagccatcgcgagttacaaaaagcttgtcggtcataaaggggcctacgagctcgaaccggaagcagaattgattgtctatccaaaaggtgacaagtcggtctatgcatatcaagtgaccggaacgatccttgaagcggaagagccatcacgttggacatacttcatcgatgcgggaacgggtgaggtactcaataaattcgaccagttggctcacgcgagaccgacgaacggtgtgacgggtactacctatacgggaacgggaatcgatgtgctcggctacagccaaacgttcaagacaacgaaaagcggatcttactattatctccaagattcaacgcgtggaaagggaatctatacgtacgatgcgaaaaaccgtacgacactcccaggttcgctatgggctgacgtggataacgtcttgaacacgacttacgaccgtgccgctgtctctgcacacgtcaacgcgacaaagacatatgatttctacaaaaacacttacggacggaacagctatgacaacgccggagctgcattaaactctacggttcactatagccgcagctacaacaatgcgttctgggatggaagtaagatggtatacggagacggagacggtcaaacgttcacgtatctttcaggtgcacttgacgtagtcgctcacgaattgactcacgcgatcacggagtacacggcaggactgatctatcaaaacgagtctggggcgatcaacgaagcagtatcggatatcctaggtacagtggcggaatatagcgtcggcactaacttcgactggttggtaggggaagacatctacacaccaggtgtagcgggcgacggacttcgttcgatggcgaacccggctgcatacggtgacccagatcactactcgaaacgttacacaggcacacaagataatggtggggttcacatcaactcaggtatcgtcaacaaagcggcatacctcttaggtaatggtggatcacactacggcgtatcggtacaaggtgtaggagtcatggcgatgggggatatctactaccgtgccctcaacgtttacttgacaccaacatctaacttctcaagccttcgtcaagcggtcgtgcaatctgcgaaagacctttacggtgcgacaagtccacaagcggtatcagctgcgaaatcatttgacgctgtcggaatctactaagttctcaacatctcttccgaaattttcggaagggattttcttatgcagtttttgggtaaatcaaggttgaacgtcgagcgaaaaggagaacgagtatgaa

SEQ ID NO: 4 - protein, 509 aa, Exiguobacterium AT1b
MKKVVSTSLIAGVLLVPQLVGAAELKSGTLTKPSESAPTTIVKEYVKSKGEFKTIESKSDKVGKVVKLQQTVDGVPVFGGVVVGVVDEAGQLKTVVDDAKSVKNLHKSIKLTEKKAIASYKKLVGHKGAYELEPEAELIVYPKGDKSVYAYQVTGTILEAEEPSRWTYFIDAGTGEVLNKFDQLAHARPTNGVTGTTYTGTGIDVLGYSQTFKTTKSGSYYYLQDSTRGKGIYTYDAKNRTTLPGSLWADVDNVLNTTYDRAAVSAHVNATKTYDFYKNTYGRNSYDNAGAALNSTVHYSRSYNNAFWDGSKMVYGDGDGQTFTYLSGALDVVAHELTHAITEYTAGLIYQNESGAINEAVSDILGTVAEYSVGTNFDWLVGEDIYTPGVAGDGLRSMANPAAYGDPDHYSKRYTGTQDNGGVHINSGIVNKAAYLLGNGGSHYGVSVQGVGVMAMGDIYYRALNVYLTPTSNFSSLRQAVVQSAKDLYGATSPQAVSAAKSFDAVGIY

SEQ ID NO: 5 - DNA, 1536 bp, Exiguobacterium sibiricum
atgaagaagttccttgctacgagccttgttgcatcagttcttgttgtaccaactgttgtaggtgcggagggccttcaagctggcaagcttactaaggcatctagcgagcctgcggcttctatcgtaaaggagtacgttaacaagaagggcgacttctctgttcaagacgttcagaaggacggctcatcaaacatcgttcgccttcaacaagaggttgacggcgttcctgtttttggcagcgttgtagttggcaacgttgctaaggacggcactcttaaggctgtagtaaacgacgcaatcaacgttaagggcaagcctggccttgcaaagaaagcttcactttctgagaagaaggcacttaagctttaccaaaaggctatcaaagcaacggagttcgaggtagcaccaaaggctgagcttgtaatctacccagtaaaggacgacgctgtttacgcttacaaggttacatctactgtacttgcaggcaaggagccttcacgctggacttacttcatcgacgcaaactctggcaaggttcttaacaagtacgaccaacttgcacatgctaagcctgttaacactgttacgggcacgacatctgttggcacaggcacgacagttcttggcactactgctacgtttaacacagtaaagtctggcagctattactaccttcaagactctactcgtggcaagggcatctacacatacgacgctaagaagcgcaacactcttcctggctctctttgggctgaccttgacaaccaattcaacacaacatacgatcgtgctgcggtttctgcacaagtaaacgctgtaaagacttatgacttctacaagaacacatatggtcgcaactcttacgacaacgctggcgctgctcttaactcatcagtacactatagcacatcttacaacaacgctttctgggacggcactaagatggtttatggcgacggcgacggctctactttcacgtatctttctggcgctcttgacgttgttgctcacgagcttactcacgctgtaacagagtatactgcaggccttgtttaccaaaacgagtctggcgctatcaacgaggctgtttcagacatcatgggcacagtagctgagtatagcgttggcagcaacttcgactggcttgtaggcgaggacatctacactcctggcgtttctggcgacgctcttcgctcaatgtctaacccagctgcgtacggcgacccagaccactattctaagcgctacactggcacacaagacaatggtggcgttcatactaactctggcatcgtaaacaaggctgcttaccttcttggcaatggtggcacgcacactggcgttacggtaacaggcgttggcgttcctaagcttggcgctatctactatcgcgctctttctgtttatcttacacctaactctaacttcagctctcttcgcgcagctgttgtacaatcagctaaggacctttacggctctacgagcgctgaggcgacagcggcagccaagtctttcgacgcagttggcgtatactaa

SEQ ID NO: 6 - DNA, 1530 bp, Exiguobacterium AT1b
atgaagaaagttgtatcaacttctcttatcgctggcgtacttcttgtacctcaacttgtaggcgcagctgagcttaagagcggcacacttacgaagccttctgagtctgcaccaactactatcgtaaaggagtacgttaagtcaaagggcgagttcaagacaatcgagtctaagtcagacaaggttggcaaggttgtaaaacttcaacagactgtagacggcgtacctgtatttggtggcgtagttgttggcgttgtagacgaggcaggccaacttaagactgtagtagacgacgcaaagtcagttaagaaccttcataagtctatcaagcttacagagaaaaaggcaatcgcaagctataagaagcttgtaggccacaaaggcgcttacgagcttgagcctgaggctgagcttatcgtatatccaaagggcgacaagtcagtatacgcataccaggtaactggcacaatccttgaggcagaggagccgagccgctggacttacttcatcgacgctggcactggcgaggtacttaacaagttcgaccagcttgctcatgctcgtccgacaaacggcgtaacaggcactacttatactggcacaggcatcgacgttcttggctactctcaaactttcaagactacaaaatctggcagctactactatcttcaggactcaactcgtggcaagggcatctatacatacgacgctaagaaccgcacaacgcttccaggctcactttgggcagacgttgacaacgttcttaacactacatacgatcgtgctgcggtatcagcgcacgttaacgcaactaagacttacgacttctataagaacacttatggtcgcaactcttatgacaatgctggcgcagcgcttaactctactgtacactattctcgcagctataacaatgcgttttgggacggctcaaagatggtttacggcgacggcgacggccaaacatttacatacctttcaggcgcacttgacgttgttgctcatgagcttactcatgctatcacggagtacacagctggccttatctatcaaaacgagtctggtgcgatcaacgaggcagtatctgacatccttggcactgttgctgagtactctgtaggcacgaacttcgactggcttgtaggcgaggacatctatactccaggcgtagctggcgacggccttcgctcaatggcgaacccagctgcttacggcgaccctgaccattactctaaacgctatacaggcacgcaagacaatggtggcgtacacatcaactcaggcatcgttaacaaggctgcatatcttcttggcaatggtggctctcattacggcgtttctgtacaaggcgttggcgtaatggcaatgggcgacatctactatcgcgcacttaacgtataccttactcctacttcaaacttttcaagccttcgccaagcggtagtacaatctgctaaggacctttatggcgcaacatctcctcaagctgtttctgcggcaaagtcttttgacgcagttggcatctactaa

SEQ ID NO: 7 - DNA, 38 bp, Artificial Sequence
  Forward primer
gttcatcgatcgcatcggctgagggccttcaagctggc

SEQ ID NO: 8 - DNA, 43 bp, Artificial Sequence
  Reverse primer
gcgtttttttattgattaacgcgtttagtatacgccaactgcg

SEQ ID NO: 9 - protein, 317 aa, Exiguobacterium sibiricum
VTGTTSVGTGTTVLGTTATFNTVKSGSYYYLQDSTRGKGIYTYDAKKRNTLPGSLWADLDNQFNTTYDRAAVSAQVNAVKTYDFYKNTYGRNSYDNAGAALNSSVHYSTSYNNAFWDGTKMVYGDGDGSTFTYLSGALDVVAHELTHAVTEYTAGLVYQNESGAINEAVSDIMGTVAEYSVGSNFDWLVGEDIYTPGVSGDALRSMSNPAAYGDPDHYSKRYTGTQDNGGVHTNSGIVNKAAYLLGNGGTHTGVTVTGVGVPKLGAIYYRALSVYLTPNSNFSSLRAAVVQSAKDLYGSTSAEATAAAKSFDAVGVY

SEQ ID NO: 10 - protein, 315 aa, Exiguobacterium sp.AT1b
GTTYTGTGIDVLGYSQTFKTTKSGSYYYLQDSTRGKGIYTYDAKNRTTLPGSLWADVDNVLNTTYDRAAVSAHVNATKTYDFYKNTYGRNSYDNAGAALNSTVHYSRSYNNAFWDGSKMVYGDGDGQTFTYLSGALDVVAHELTHAITEYTAGLIYQNESGAINEAVSDILGTVAEYSVGTNFDWLVGEDIYTPGVAGDGLRSMANPAAYGDPDHYSKRYTGTQDNGGVHINSGIVNKAAYLLGNGGSHYGVSVQGVGVMAMGDIYYRALNVYLTPTSNFSSLRQAVVQSAKDLYGATSPQAVSAAKSFDAVGIY

SEQ ID NO: 11 - protein, 7 aa, Artificial Sequence
  N terminal
VTGTTSV

SEQ ID NO: 12 - protein, 7 aa, Artificial Sequence
  N terminal AT1b
GTTYTGT

SEQ ID NO: 13 - protein, 300 aa, Bacillus amyloliquefaciens
AATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 14 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2016/0145540 A1
(43) Pub. Date: May 26, 2016

(54) Detergent Compositions Comprising Metalloproteases

(71) Applicant: Novozymes A/S, Bagsvaerd (DK)

(72) Inventors: Astrid Benie, Vaerloese (DK); Peter Rahbek Oestergaard, Virum (DK); Morten Gjermansen, Greve (DK); Martin Simon Borchert, Hilleroed (DK)

(21) Appl. No.: 14/421,066

(22) PCT Filed: Aug. 21, 2013
(86) PCT No.: PCT/EP2013/067410

§ 371 Date: Feb. 11, 2015

(30) Foreign Application Priority Data
Aug. 22, 2012 (EP) 12181350.5

(51) Int. Cl. C11D 3/386; C12N 9/52

(52) U.S. Cl. CPC C11D 3/386; C12N 9/52; C12Y 304/24

(57) ABSTRACT

The present invention relates to the use of and compositions comprising isolated polypeptides having protease activity and polynucleotides encoding the polypeptides. The invention also relates to cleaning compositions, and the use of the polypeptides in cleaning processes.

### REFERENCE TO A SEQUENCE LISTING

This application contains a Sequence Listing in computer readable form, which is incorporated herein by reference.

### FIELD OF THE INVENTION

The present invention relates to cleaning and/or detergent compositions comprising metalloproteases (E.C 3.4.24). The invention further concerns the use of the metalloproteases in cleaning processes, such as dish wash and laundry. Further the invention concerns methods of doing cleaning, such as dish wash and laundry.

### DESCRIPTION OF THE RELATED ART

The detergent industry has for more than 30 years implemented different enzymes in detergent formulations, most commonly used enzymes includes proteases, amylases and lipases each adapted for removing various types of stains. In addition to the enzymes detergent compositions typically include a complex combination of ingredients. For example, most cleaning products include surfactant system, bleaching agents or builders. Despite the complexity of current detergents, there remains a need for developing new detergent compositions comprising new enzymes and/or enzyme blends.

Metalloproteases are proteolytic enzymes having an absolute requirement for metal ion for their activity. Most metalloproteases are zinc-dependent, although some use other transition metals. Metalloproteases have been widely used in different industries like food and brewing industry. One of the most well characterized groups of metalloproteases is the thermolysins. The thermolysins belong to the M4 family metalloproteases and have been used, e.g., in peptide synthesis processes. For such applications the thermolysins need to be active at high temperatures and focus has been on increasing their performance at high temperatures. In WO 2004/011619 (Stratagene) thermostabile variants of thermolysin-like protease with altered cleavage specificity is described. Another M4 metalloprotease is the Bacillus amyloliquefaciens metalloprotease, also known as Neutrase®, which has been used for many years as an additive in various food and feed products and, e.g., in brewing. This metalloprotease has also been described for use in detergent and cleaning compositions and processes as described, e.g., in WO 2007/044993, use of storage-stable metalloproteases in detergent or WO 2009/058518, and EP 1 288 282 (Unilever), which describes a blend of a metalloprotease and a serine protease for use in dish washing. The use of Thermolysin like metalloproteases in detergents is also described in WO20099058303 (Danisco US INC.).

However, the use of metalloproteases in the detergent industry has been very limited and focus has been on the use of the metalloproteases Neutrase® and/or “NprE” as set forth in WO 2007/044993. Generally, metalloproteases are very unstable under conventional wash conditions and in conventional detergent compositions. Thus, the use of metalloproteases in wash and cleaning processes and in detergents has been limited.

The increased focus on improving the washing processes in order to make them more environmental friendly has resulted in a global tendency to lowering wash time, pH and temperature, decreasing the amount of detergent components which may influence the environment negatively. The present invention is directed to these and other important ends.

### SUMMARY OF THE INVENTION

One aspect of the invention relates to a composition comprising a polypeptide having protease activity wherein the polypeptide having protease activity is selected among:
a) a polypeptide having at least 70% sequence identity, such as at least 75% identity, such as at least 80% identity, such as at least 85% sequence identity, such as at least 90% identity, such as at least 95% identity, such as at least 97% identity, such as at least 98% identity, such as at least 99% identity to the mature polypeptide of SEQ ID NO: 2 or SEQ ID NO 4;
(b) a polypeptide encoded by a polynucleotide having at least 70% sequence identity, such as at least 75% identity, such as at least 80% identity, such as at least 85% sequence identity, such as at least 90% identity, such as at least 95% identity, such as at least 97% identity, such as at least 98% identity and such as at least 99% identity to the mature polypeptide coding sequence of SEQ ID NO: 1 or SEQ ID NO 3;
(c) a variant comprising a substitution, deletion, and/or insertion of one or more (several) amino acids of the mature polypeptide of SEQ ID NO: 2 or SEQ ID NO 3; and
(d) a fragment of a polypeptide of (a), (b) or (c) that has protease activity.

In a particular aspect of the invention the composition is a cleaning composition such as a detergent composition or a detergent additive. Furthermore, the invention relates to the use of a composition according to the invention in a cleaning process, such as laundry, hard surface cleaning, dish wash or automated dish wash.

Another aspect of the invention relates to the use of a polypeptide having protease activity in a cleaning process, wherein the polypeptide having protease activity is selected among:

a) a polypeptide having at least 70% sequence identity, such as at least 75% identity, such as at least 80% identity, such as at least 85% sequence identity, such as at least 90% identity, such as at least 95% identity, such as at least 97% identity, such as at least 98% identity and such as at least 99% identity to the mature polypeptide of SEQ ID NO: 2 or SEQ ID NO 4;

(b) a polypeptide encoded by a polynucleotide having at least 70% sequence identity, such as at least 75% identity, such as at least 80% identity, such as at least 85% sequence identity, such as at least 90% identity, such as at least 95% identity, such as at least 97% identity, such as at least 98% identity and most preferred at least 99% identity to the mature polypeptide coding sequence of SEQ ID NO: 1 or SEQ ID NO 3;

(c) a variant comprising a substitution, deletion, and/or insertion of one or more (several) amino acids of the mature polypeptide of SEQ ID NO: 2 or SEQ ID NO 4; and (d) a fragment of a polypeptide of (a), (b) or (c) that has protease activity.

The isolated polypeptide of the invention is a metalloprotease belonging to the M4 metalloprotease group. Preferably the isolated polypeptide is derived from Exiguobacterium.

The present invention relates to the use of the metalloproteases in particular metalloprotease of the M4 type such as metalloproteases having at least 70% identity to SEQ ID NO 2 or SEQ ID NO 4 in cleaning processes, such as laundry and dish wash, and in particular to the use in low temperature. The invention also relates to detergent compositions and cleaning compositions comprising the metalloproteases.

A particular aspect of the invention relates to a method of cleaning, said method comprising the steps of: contacting a surface with a metalloprotease according to the invention.

Another particular aspect, the invention relates to a composition such as a detergent composition comprising a metalloprotease and a surfactant.

Yet another particular aspect, the invention relates to a method for removing a stain from a surface which comprises contacting the surface with a composition according to the invention.

### Overview of Sequence Listing

SEQ ID NO 1: is the DNA sequence of Exiguobacterium sibiricum.

SEQ ID NO 2: is the amino acid sequence as deduced from SEQ ID NO: 1 (SWISSPROT: B1YFR1).

SEQ ID NO 3: is the DNA sequence of Exiguobacterium sp. AT1b

SEQ ID NO 4: is the amino acid sequence as deduced from SEQ ID NO: 3 (SWISSPROT: C4L1B3).

SEQ ID NO 5: DNA encoding the metalloprotease Exiguobacterium sibiricum with a codon usage optimized for expression in a Bacillus strain.

SEQ ID NO 6: DNA encoding the metalloprotease Exiguobacterium sp. AT1b with a codon usage optimized for expression in a Bacillus strain.

SEQ ID NO 7: Forward primer sequence

SEQ ID NO 8: Reverse primer sequence

SEQ ID NO 9: Mature polypeptide of Exiguobacterium sibiricum

SEQ ID NO 10: Mature polypeptide of Exiguobacterium sp.AT1b

SEQ ID NO 11: N-terminal sequences of Exiguobacterium sibiricum

SEQ ID NO 12: N-terminal sequence of Exiguobacterium sp.AT1b

SEQ ID NO 13: Bacillus amyloliquefaciens protease Uniprot: P06832 (Neutrase®)

SEQ ID NO 14: Bacillus clausii protease (Savinase®)

### DEFINITIONS

“Polypeptides having protease activity” polypeptides having protease activity, or proteases, are sometimes also designated peptidases, proteinases, peptide hydrolases, or proteolytic enzymes. Proteases may be of the exo-type that hydrolyses peptides starting at either end thereof, or of the endo-type that act internally in polypeptide chains (endopeptidases). Endopeptidases show activity on N- and C-terminally blocked peptide substrates that are relevant for the specificity of the protease in question.

The term “protease” is defined herein as an enzyme that hydrolyses peptide bonds. The present invention provides for the use of polypeptides having protease activity in detergent compositions. It also provides polynucleotides encoding the polypeptides. The proteases of the invention are metalloproteases of the MEOPS family M4. The polypeptides of the present invention have at least 20%, e.g., at least 40%, at least 50%, at least 60%, at least 70%, at least 80%, at least 90%, at least 95%, at least 96%, at least 97%, at least 98%, at least 99%, and at least 100% of the protease activity of the mature polypeptide of SEQ ID NO: 2 or SEQ ID NO: 4.

“Protease activity” can be measured using any assay, in which a substrate is employed, that includes peptide bonds relevant for the specificity of the protease in question. Assay-pH and assay-temperature are likewise to be adapted to the protease in question. Examples of assay-pH-values are pH 2, 3, 4, 5, 6, 7, 8, 9, 10, 11, or 12. Examples of assay-temperatures are 15, 20, 25, 30, 35, 37, 40, 45, 50, 55, 60, 65, 70, 80, 90, or 95° C. Examples of general protease substrates are collagen, casein, bovine serum albumin and hemoglobin. In the classical Anson and Mirsky method, denatured hemoglobin is used as substrate and after the assay incubation with the protease in question, the amount of trichloroacetic acid soluble hemoglobin is determined as a measurement of protease activity (Anson, M. L. and Mirsky, A. E., 1932, J. Gen. Physiol. 16: 59 and Anson, M. L., 1938, J. Gen. Physiol. 22: 79).

For the purpose of the present invention, protease activity was determined using assays which are described in “Materials and Methods”, such as the Protazyme OL assay.

The term “metalloprotease” as used herein refers to a protease having one or more metal ions in the binding/active site.

The term “M4 metalloprotease Family” or “M4 metalloprotease” or “M4” as used herein means a polypeptide falling into the M4 metalloprotease family according to Rawlings et al., Biochem. J., 290, 205-218 (1993) and as further described in MEROPS—(Rawlings et al., MEROPS: the peptidase database, Nucl Acids Res, 34 Database issue, D270-272, 2006). The M4 metalloproteases are neutral metalloproteases containing mainly endopeptidases. All peptidases in the family bind a single, catalytic zinc ion. M4 metalloprotease family members include the common HEXXH motif, where the histidine residues serve as zinc ligands and glutamate is an active site residue. M4 metalloproteases have a pH optimum mainly at neutral pH. The M4 metalloprotease family includes, e.g., Neutrase® (classified as MEROPS subclass M04.014), Thermolysin, Bacillolysin, vibriolysin, pseudolysin, Msp peptidase, coccolysin, aureolysin, vimelysin, lambda toxin neutral peptidase B, PA peptidase (Aeromonas-type), griselysin, stearolysin, MprIII (Alteromonas sp. strain 0-7), pap6 peptidase, neutral peptidase (Thermoactinomyces-type), ZmpA peptidase (Burkholderia sp.), zpx peptidase, PrtS peptidase (Photorhabdus luminescens), protealysin, ZmpB peptidase (Burkholderia sp.). The M4 metalloprotease family of polypeptides has been further characterized and presently includes, according to MEROPS, at least twenty-two subclasses for which a distinct MEROPS ID (i.e., an identifier of the formula M04.xxx) has been assigned, as well as non-peptidase homologues and unassigned peptidases.

The term “isolated polypeptide” as used herein refers to a polypeptide that is isolated from a source. In one aspect, the variant or polypeptide is at least 20% pure, more preferably at least 40% pure, more preferably at least 60% pure, even more preferably at least 80% pure, most preferably at least 90% pure and even most preferably at least 95% pure, as determined by SDS-PAGE.

The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form. This can be accomplished, for example, by preparing the variant or polypeptide by well-known recombinant methods or by classical purification methods.

The term “mature polypeptide coding sequence” means a polynucleotide that encodes a mature polypeptide having protease activity.

The relatedness between two amino acid sequences or between two nucleotide sequences is described by the parameter “sequence identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.orq), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

The term “fragment” means a polypeptide having one or more (several) amino acids deleted from the amino and/or carboxyl terminus of a mature polypeptide; wherein the fragment has protease activity.

The term “functional fragment of a polypeptide” or “functional fragment thereof” is used to describe a polypeptide which is derived from a longer polypeptide, e.g., a mature polypeptide, and which has been truncated either in the N-terminal region or the C-terminal region or in both regions to generate a fragment of the parent polypeptide. To be a functional polypeptide the fragment must maintain at least 20%, preferably at least 40%, more preferably at least 50%, more preferably at least 60%, more preferably at least 70%, more preferably at least 80%, even more preferably at least 90%, most preferably at least 95%, and even most preferably at least 100% of the protease activity of the full-length/mature polypeptide. An M4 metalloprotease may be truncated such that certain domain is removed to generate a functional fragment, which may be polypeptides where less than 200 amino acids have been removed from the mature M4 Metalloprotease, preferably less than 150 amino acids, more preferably less than 120, 100, 80, 60, 40, 30 amino acids, even more preferably less than 20 amino acids and most preferably less than 10 amino acids have been removed from the mature polypeptide.

The term “subsequence” means a polynucleotide having one or more (several) nucleotides deleted from the 5′ and/or 3′ end of a mature polypeptide coding sequence; wherein the subsequence encodes a fragment having protease activity.

The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “variant” means a polypeptide having protease activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1-3 amino acids adjacent to an amino acid occupying a position.

The terms “cleaning compositions” and “cleaning formulations,” refer to compositions that find use in the removal of undesired compounds from items to be cleaned, such as fabric, carpets, dishware including glassware, contact lenses, hard surfaces such as tiles, zincs, floors, and table surfaces, hair (shampoos), skin (soaps and creams), teeth (mouthwashes, toothpastes), etc. The terms encompasses any materials/compounds selected for the particular type of cleaning composition desired and the form of the product (e.g., liquid, gel, granule, or spray compositions), as long as the composition is compatible with the metalloproteases and other enzyme(s) used in the composition. The specific selection of cleaning composition materials is readily made by considering the surface, item or fabric to be cleaned, and the desired form of the composition for the cleaning conditions during use. These terms further refer to any composition that is suited for cleaning, bleaching, disinfecting, and/or sterilizing any object and/or surface. It is intended that the terms include, but are not limited to detergent composition (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

The term “detergent composition”, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types.

The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. It is intended that in addition to the metalloproteases according to the invention, the term encompasses detergents that contains, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

The term “fabric” encompasses any textile material. Thus, it is intended that the term encompass garments, as well as fabrics, yarns, fibers, non-woven materials, natural materials, synthetic materials, and any other textile material.

The term “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers. The term, “textile materials” is a general term for fibers, yarn intermediates, yarn, fabrics, and products made from fabrics (e.g., garments and other articles).

The term “non-fabric detergent compositions” include non-textile surface detergent compositions, including but not limited to dishwashing detergent compositions, oral detergent compositions, denture detergent compositions, and personal cleansing compositions.

The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like. The term “effective amount” of a metalloprotease refers to the quantity of metalloproteases described hereinbefore that achieves a desired level of enzymatic activity, e.g., in a defined detergent composition.

The term “wash performance” of an enzyme refers to the contribution of an enzyme to washing that provides additional cleaning performance to the detergent without the addition of the enzyme to the composition. Wash performance is compared under relevant washing conditions. Wash performance of enzymes is conveniently measured by their ability to remove certain representative stains under appropriate test conditions. In these test systems, other relevant factors, such as detergent composition, detergent concentration, water hardness, washing mechanics, time, pH, and/or temperature, can be controlled in such a way that conditions typical for household application in a certain market segment are imitated.

The term “water hardness” or “degree of hardness” or “dH” or “° dH” as used herein refers to German degrees of hardness. One degree is defined as 10 milligrams of calcium oxide per liter of water.

The term “relevant washing conditions” is used herein to indicate the conditions, particularly washing temperature, time, washing mechanics, detergent concentration, type of detergent and water hardness, actually used in households in a detergent market segment.

The term “improved property” is used to indicate that a better end result is obtained in a property compared to the same process performed without the enzyme. Exemplary properties which are preferably improved in the processes of the present invention include wash performance, enzyme stability, enzyme activity and substrate specificity.

The term “improved wash performance” is used to indicate that a better end result is obtained in stain removal from items washed (e.g., fabrics or dishware and/or cutlery) under relevant washing conditions as compared to no enzyme or to a reference enzyme, or that less enzyme, on weight basis, is needed to obtain the same end result relative to no enzyme or to a reference enzyme. Improved wash performance could in this context also be that the same effect, e.g., stain removal effect is obtained in shorter wash time, e.g., the enzymes provide their effect more quickly under the tested conditions.

The term “retained wash performance” is used to indicate that the wash performance of an enzyme, on weight basis, is at least 80 percent relative to another enzyme under relevant washing conditions.

The term “enzyme detergency” or “detergency” or “detergency effect” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process an effect that also is termed anti-redeposition, restoring fully or partly the whiteness of textiles, which originally were white but after repeated use and wash have obtained a grayish or yellowish appearance an effect that also is termed whitening. Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric an effect that is also termed dye transfer inhibition or anti-back staining, removal of protruding or broken fibers from a fabric surface to decrease pilling tendencies or remove already existing pills or fuzz an effect that also is termed anti-pilling, improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

The term “anti-redeposition” as used herein describes the reduction or prevention of redeposition of soils dissolved or suspended in the wash liquor onto the cleaned objects. Redeposition may be seen after one or multiple washing cycles (e.g., as a greying, yellowing or other discolorations).

The term “adjunct materials” means any liquid, solid or gaseous material selected for the particular type of detergent composition desired and the form of the product (e.g., liquid, granule, powder, bar, paste, spray, tablet, gel, or foam composition), which materials are also preferably compatible with the metalloprotease enzyme used in the composition. In some embodiments, granular compositions are in “compact” form, while in other embodiments, the liquid compositions are in a “concentrated” form.

The term “stain removing enzyme” as used herein, describes an enzyme that aids the removal of a stain or soil from a fabric or a hard surface. Stain removing enzymes act on specific substrates, e.g., protease on protein, amylase on starch, lipase and cutinase on lipids (fats and oils), pectinase on pectin and hemicellulases on hemicellulose. Stains are often depositions of complex mixtures of different components which either results in a local discoloration of the material by itself or which leaves a sticky surface on the object which may attract soils dissolved in the washing liquor thereby resulting in discoloration of the stained area. When an enzyme acts on its specific substrate present in a stain the enzyme degrades or partially degrades its substrate thereby aiding the removal of soils and stain components associated with the substrate during the washing process. For example, when a protease acts on a grass stain it degrades the protein components in the grass and allows the green/brown colour to be released during washing.

The term “reduced amount” means in this context that the amount of the component is smaller than the amount which would be used in a reference process under otherwise the same conditions. In a preferred embodiment the amount is reduced by, e.g., at least 5%, such as at least 10%, at least 15%, at least 20% or as otherwise herein described.

The term “low detergent concentration” system includes detergents where less than about 800 ppm of detergent components is present in the wash water. Asian, e.g., Japanese detergents are typically considered low detergent concentration systems.

The term “medium detergent concentration” system includes detergents wherein between about 800 ppm and about 2000 ppm of detergent components is present in the wash water. North American detergents are generally considered to be medium detergent concentration systems.

The term “high detergent concentration” system includes detergents wherein greater than about 2000 ppm of detergent components is present in the wash water. European detergents are generally considered to be high detergent concentration systems.

### DETAILED DESCRIPTION OF THE INVENTION

### Polypeptides Having Protease Activity

In an embodiment, the present invention relates to a composition such as a cleaning composition, comprising an isolated polypeptides having a sequence identity to the mature polypeptide of SEQ ID NO: 2 or SEQ ID NO: 4 of at least 70%, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have protease activity.

Thus an embodiment of the invention relates to a composition comprising a polypeptide having protease activity wherein the polypeptide having protease activity is selected among:
a) a polypeptide having at least 70% sequence identity, such as at least 75% identity, such as at least 80% identity, such as at least 85% sequence identity, such as at least 90% identity, such as at least 95% identity, such as at least 97% identity, such as at least 98% identity, such as at least 99% identity to the mature polypeptide of SEQ ID NO: 2 or SEQ ID NO 4;
(b) a polypeptide encoded by a polynucleotide having at least 70% sequence identity, such as at least 75% identity, such as at least 80% identity, such as at least 85% sequence identity, such as at least 90% identity, such as at least 95% identity, such as at least 97% identity, such as at least 98% identity and such as at least 99% identity to the mature polypeptide coding sequence of SEQ ID NO: 1 or SEQ ID NO 3;
(c) a variant comprising a substitution, deletion, and/or insertion of one or more (several) amino acids of the mature polypeptide of SEQ ID NO: 2 or SEQ ID NO 4; and
(d) a fragment of a polypeptide of (a), (b) or (c) that has protease activity.
   In a particular aspect of the invention the composition is cleaning composition such as a detergent composition or a detergent additive.

In another embodiment, the present invention relates to the use of isolated polypeptides having a sequence identity to the mature polypeptide of SEQ ID NO: 2 or SEQ ID NO: 4 of at least 70%, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have protease activity in a cleaning process or in a detergent.

Thus another aspect of the invention relates to the use of a polypeptide having protease activity in a cleaning process, wherein the polypeptide having protease activity is selected among:

a) a polypeptide having at least 70% sequence identity, such as at least 75% identity, such as at least 80% identity, such as at least 85% sequence identity, such as at least 90% identity, such as at least 95% identity, such as at least 97% identity, such as at least 98% identity and such as at least 99% identity to the mature polypeptide of SEQ ID NO: 2 or SEQ ID NO 4;

(b) a polypeptide encoded by a polynucleotide having at least 70% sequence identity, such as at least 75% identity, such as at least 80% identity, such as at least 85% sequence identity, such as at least 90% identity, such as at least 95% identity, such as at least 97% identity, such as at least 98% identity and most preferred at least 99% identity to the mature polypeptide coding sequence of SEQ ID NO: 1 or SEQ ID NO 3;

(c) a variant comprising a substitution, deletion, and/or insertion of one or more (several) amino acids of the mature polypeptide of SEQ ID NO: 2 or SEQ ID NO 4; and

(d) a fragment of a polypeptide of (a), (b) or (c) that has protease activity.

In one aspect, the polypeptides differ by no more than 10 amino acids, e.g., 1, 2, 3, 4, 5, 6, 7, 8, or 9, from the mature polypeptide of SEQ ID NO: 2.

In one aspect, the polypeptides differ by no more than 10 amino acids, e.g., 1, 2, 3, 4, 5, 6, 7, 8, or 9, from the mature polypeptide of SEQ ID NO: 4.

In one aspect, the polypeptide for use in the present invention such as comprises or consists of the amino acid sequence of SEQ ID NO: 2 or an allelic variant thereof; or is a fragment thereof having protease activity. In another aspect, the polypeptide comprises or consists of the mature polypeptide of SEQ ID NO: 2. In another aspect, the polypeptide comprises or consists of amino acids 1 to 317 of SEQ ID NO: 2.

In one aspect, the polypeptide for use in the present invention such as comprises or consists of the amino acid sequence of SEQ ID NO: 4 or an allelic variant thereof; or is a fragment thereof having protease activity. In another aspect, the polypeptide comprises or consists of the mature polypeptide of SEQ ID NO: 4. In another aspect, the polypeptide comprises or consists of amino acids 1 to 315 of SEQ ID NO: 4.

In a preferred embodiment of the invention the isolated polypeptide of the invention is a metalloprotease belonging to the M4 metalloprotease group. Preferably the isolated polypeptide is derived from Exiguobacterium. More preferably the polypeptide is derived from Exiguobacterium sibiricum or Exiguobacterium sp. AT1b. Thus the present invention relates to the use of the metalloproteases in particular metalloprotease of the M4 type such as metalloprotease having at least 70% identity to SEQ ID NO 2 or SEQ ID NO 4 in cleaning processes, such as laundry or dish wash. The invention also relates to detergent compositions and cleaning compositions comprising the metalloproteases.

Metalloproteases of the M4 type is metalloproteases belonging to the M4 metalloprotease Family, M4 metalloprotease or simply M4 as used herein the term means a polypeptide falling into the M4 metalloprotease family according to Rawlings et al., Biochem. J., 290, 205-218 (1993) and as further described in MEROPS—(Rawlings et al., MEROPS: the peptidase database, Nucl Acids Res, 34 Database issue, D270-272, 2006). The M4 metalloproteases are neutral metalloproteases containing mainly endopeptidases. All peptidases in the family bind a single, catalytic zinc ion. M4 metalloprotease family members include the common HEXXH motif, where the histidine residues serve as zinc ligands and glutamate is an active site residue. M4 metalloproteases have a pH optimum mainly at neutral pH. The M4 metalloprotease family of polypeptides has been further characterized and presently includes, according to MEROPS, at least twenty-two subclasses for which a distinct MEROPS ID (i.e., an identifier of the formula M04.xxx) has been assigned, as well as non-peptidase homologues and unassigned peptidases.

In another embodiment, the present invention relates to the use of an isolated polypeptide having protease activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1 or SEQ ID NO: 3, or (ii) the full-length complement of (i) or (ii) (Sambrook et al., 1989, Molecular Cloning, A Laboratory Manual, 2d edition, Cold Spring Harbor, New York).

The polynucleotide of SEQ ID NO: 1 or SEQ ID NO: 3 or a subsequence thereof, as well as the polypeptide of SEQ ID NO: 2 or SEQ ID NO: 4 or a fragment thereof, may be used to design nucleic acid probes to identify and clone DNA encoding polypeptides having protease activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Such as, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵5, biotin, or avidin). Such probes are encompassed by the present invention.

A genomic DNA or cDNA library prepared from such other strains may be screened for DNA that hybridizes with the probes described above and encodes a polypeptide having protease activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, SEQ ID NO: 3 or a subsequence thereof, the carrier material is used in a Southern blot.

For purposes of the present invention, hybridization indicates that the polynucleotide hybridizes to a labeled nucleic acid probe corresponding to (i) SEQ ID NO: 1 or SEQ ID NO: 3; (ii) the mature polypeptide coding sequence of SEQ ID NO: 1 or SEQ ID NO: 3; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In another embodiment, the present invention relates to an isolated polypeptide having protease activity encoded by a polynucleotide having a sequence identity to the mature polypeptide coding sequence of SEQ ID NO: 1 or SEQ ID NO 3 of at least 70%, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In another embodiment, the present invention relates to variants of the mature polypeptide of SEQ ID NO: 2 or variants of the mature polypeptide of SEQ ID NO: 4 comprising a substitution, deletion, and/or insertion at one or more (e.g., several) positions. In an embodiment, the number of amino acid substitutions, deletions and/or insertions introduced into the mature polypeptide of SEQ ID NO: 2 or SEQ ID NO: 4 is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, AlaNal, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like. Examples of positions in M4 metalloproteases which preferably could be altered: 110, 114, 115, 116, 119, 133, 144, 150, 202, 211, 225, 227. In particular, the following specific mutations lead to increased probabilities e.g. increased activity in M4 metalloproteases: Y110W, F114A, N116D, Q119R,E,D,H,M,S,G,A, F133L, L144S, D150N,H,W, L202 F,Y,G,A,V, Y211W, Q225R, N227H (numbering according to the mature peptide of the M4 protease from Bacillus thermoproteolyticus, Public accession number: Uniprot: P00800, Handbook of proteolytic enzymes. 2^{nd} edition Edited by Barret A., Rawlings N., Woessner F, Elsevier Academic Press 2004).

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708 The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. In the metalloprotease Exiguobacterium sibiricum the active site residues are E144 and H232 (numbering according to SEQ ID NO 2). In the metalloprotease Exiguobacterium sp. AT1b the active site residues are E142 and H230 (numbering according to SEQ ID NO 4).

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The total number of amino acid substitutions, deletions and/or insertions of the mature polypeptide of SEQ ID NO: 2 or 4 are not more than 10, e.g., 1, 2, 3, 4, 5, 6, 7, 8 or 9.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of M4 Metalloproteases

A M4 metalloprotease useful in the present invention may be obtained from microorganisms of any genus. For purposes of the present invention, the term “obtained from” as used herein in connection with a given source shall mean that the polypeptide encoded by a nucleotide sequence is produced by the source in which it is naturally present or by a strain in which the nucleotide sequence from the source has been inserted. In a preferred aspect, the polypeptide obtained from a given source is secreted extracellularly.

A polypeptide of the present invention may be a bacterial polypeptide. For example, the polypeptide may be a gram-positive bacterial polypeptide such as an Exiguobacterium, Bacillus, Clostridium, Enterococcus, Geobacillus, Lactobacillus, Lactococcus, Oceanobacillus, Staphylococcus, Streptococcus, or Streptomyces polypeptide having protease activity, or a gram-negative bacterial polypeptide such as a Campylobacter, E. coli, Flavobacterium, Fusobacterium, Helicobacter, Ilyobacter, Neisseria, Pseudomonas, Salmonella, Ureaplasma polypeptide.

In one aspect the polypeptide is an Exiguobacterium polypeptide, such as a polypeptide from Exiguobacterium sibiricum or Exiguobacterium sp. ATM polypeptide.

In another aspect, the polypeptide is a Bacillus alkalophilus, Bacillus amyloliquefaciens, Bacillus brevis, Bacillus cereus, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Geobacillus caldolyticus, Geobacillus stearothermophilus polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

A polypeptide of the present invention may also be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Chaetomium, Cryptococcus, Filibasidium, Fusarium, Humicola, Magnaporthe, Mucor, Myceliophthora, Neocallimastix, Neurospora, Paecilomyces, Penicillium, Piromyces, Poronia, Schizophyllum, Talaromyces, Thermoascus, Thielavia, Tolypocladium, Trichoderma or Verticillium polypeptide.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another preferred aspect, the polypeptide is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus terreus, Chaetomium globosum, Coprinus cinereus, Diplodia gossyppina, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Magnaporthe grisea, Mucor miehei, Myceliophthora thermophila, Neurospora crassa, Penicillium purpurogenum, Phanerochaete chrysosporium, Poronia punctata, Pseudoplectania nigrella, Thermoascus aurantiacus, Thielavia terrestris, Trichoderma harzianum, Trichoderma koningii, Trichoderma longibrachiatum, Trichoderma reesei, Trichoderma viride, Trichophaea saccata or Verticillium tenerum polypeptide.

In a preferred aspect, the polypeptide is an Exiguobacterium sp. AT1b polypeptide, such as a polypeptide with SEQ ID NO: 4 or a polypeptide having at least 70% identity hereto. In another preferred aspect the polypeptide is an Exiguobacterium polypeptide, such as an Exiguobacterium sibiricum polypeptide, such as a polypeptide with SEQ ID NO: 2 or a polypeptide having at least 70% identity hereto.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of such a microorganism. Once a polynucleotide sequence encoding a polypeptide has been detected with the probe(s), the polynucleotide can be isolated or cloned by utilizing techniques which are well known to those of ordinary skill in the art (see, e.g., Sambrook et al., 1989, supra).

Polypeptides of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

### Compositions

The present invention also relates to compositions comprising a metalloprotease of the invention. Preferably, the compositions are enriched in a metalloprotease of the invention. The term “enriched” indicates that the protease activity of the composition has been increased.

In one embodiment, the present invention relates to compositions in particular to cleaning compositions and/or detergent compositions comprising a metalloprotease of the invention and a suitable carrier and/or excipient.

In one embodiment, the detergent composition may be adapted for specific uses such as laundry, in particular household laundry, dish washing or hard surface cleaning.

The detergent compositions of the invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. The detergent compositions of the invention may find use in hard surface cleaning, automatic dishwashing applications, as well as cosmetic applications such as dentures, teeth, hair and skin.

In a preferred embodiment, the detergent compositions comprise one or more conventional carrier(s) and/or excipient(s) such as those exemplified below.

The detergent composition of the invention may be in any convenient form, e.g., a bar, a tablet, a powder, a granule, a paste or a liquid. A liquid detergent may be aqueous, typically containing up to 70% water and 0-30% organic solvent, or non-aqueous.

Unless otherwise noted, all component or composition levels provided herein are made in reference to the active level of that component or composition, and are exclusive of impurities, for example, residual solvents or by-products, which may be present in commercially available sources.

The metalloproteases of the invention are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.75% of enzyme protein by weight of the composition, even more preferably at a level of from 0.001% to 0.5% of enzyme protein by weight of the composition.

Furthermore, the metalloproteases of the invention are normally incorporated in the detergent composition in such amounts that their concentration in the wash water is at a level of from 0.0000001% to 1% enzyme protein, preferably at a level of from 0.000005% to 0.01% of enzyme protein, more preferably at a level of from 0.000001% to 0.005% of enzyme protein, even more preferably at a level of from 0.00001% to 0.001% of enzyme protein in wash water.

As is well known, the amount of enzyme will also vary according to the particular application and/or as a result of the other components included in the compositions.

A composition for use in automatic dishwash (ADW), for example, may include 0.001%-50%, such as 0.01%-25%, such as 0.02%-20%, such as 0.1-15% of enzyme protein by weight of the composition.

A composition for use in laundry granulation, for example, may include 0.0001%-50%, such as 0.001%-20%, such as 0.01%-15%, such as 0.05%-10% of enzyme protein by weight of the composition.

A composition for use in laundry liquid, for example, may include 0.0001%-10%, such as 0.001-7%, such as 0.1%-5% of enzyme protein by weight of the composition.

In some preferred embodiments, the detergent compositions provided herein are typically formulated such that, during use in aqueous cleaning operations, the wash water has a pH of from about 5.0 to about 11.5, or in alternative embodiments, even from about 6.0 to about 10.5, such as from about 5 to about 11, from about 5 to about 10, from about 5 to about 9, from about 5 to about 8, from about 5 to about 7, from about 6 to about 11, from about 6 to about 10, from about 6 to about 9, from about 6 to about 8, from about 6 to about 7, from about 7 to about 11, from about 7 to about 10, from about 7 to about 9, or from about 7 to about 8. In some preferred embodiments, granular or liquid laundry products are formulated such that the wash water has a pH from about 5.5 to about 8. Techniques for controlling pH at recommended usage levels include the use of buffers, alkalis, acids, etc., and are well known to those skilled in the art.

Enzyme components weights are based on total active protein. All percentages and ratios are calculated by weight unless otherwise indicated. All percentages and ratios are calculated based on the total composition unless otherwise indicated. In the exemplified detergent composition, the enzymes levels are expressed by pure enzyme by weight of the total composition and unless otherwise specified, the detergent ingredients are expressed by weight of the total composition.

The enzymes of the present invention also find use in detergent additive products. A detergent additive product comprising a metalloprotease of the invention is ideally suited for inclusion in a wash process when, e.g., temperature is low, the pH is between 6 and 8 and the washing time short, e.g., below 30 min.

The detergent additive product may be a metalloprotease of the invention and preferably an additional enzyme. In one embodiment, the additive is packaged in dosage form for addition to a cleaning process. The single dosage may comprise a pill, tablet, gelcap or other single dosage unit including powders and/or liquids. In some embodiments, filler and/or carrier material(s) are included, suitable filler or carrier materials include, but are not limited to, various salts of sulfate, carbonate and silicate as well as talc, clay and the like. In some embodiments filler and/or carrier materials for liquid compositions include water and/or low molecular weight primary and secondary alcohols including polyols and diols. Examples of such alcohols include, but are not limited to, methanol, ethanol, propanol and isopropanol.

In one particularly preferred embodiment the metalloproteases according to the invention is employed in a granular composition or liquid, the metalloprotease may be in form of an encapsulated particle. In one embodiment, the encapsulating material is selected from the group consisting of carbohydrates, natural or synthetic gums, chitin and chitosan, cellulose and cellulose derivatives, silicates, phosphates, borates, polyvinyl alcohol, polyethylene glycol, paraffin waxes and combinations thereof.

The compositions according to the invention typically comprise one or more detergent ingredients. The term detergent compositions include articles and cleaning and treatment compositions. The term cleaning composition includes, unless otherwise indicated, tablet, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use. The composition can also be in unit dose packages, including those known in the art and those that are water soluble, water insoluble and/or water permeable.

In embodiments in which cleaning and/or detergent components may not be compatible with the metalloprotease of the present invention, suitable methods may be used for keeping the cleaning and/or detergent components and the metalloprotease separated (i.e., not in contact with each other) until combination of the two components is appropriate. Such separation methods include any suitable method known in the art (e.g., gelcaps, encapsulation, tablets, physical separation).

As mentioned when the metalloproteases of the invention is employed as a component of a detergent composition (e.g., a laundry washing detergent composition, or a dishwashing detergent composition), it may, for example, be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 (both to Novo Industri NS) and may optionally be coated by methods known in the art. Examples of waxy coating materials are poly (ethylene oxide) products (polyethyleneglycol, PEG) with mean molecular weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), Nickel (II), and oxovanadium (IV)). The enzymes of the detergent compositions of the invention may also be stabilized using conventional stabilizing agents such as polyol, e.g., propylene glycol or glycerol, a sugar or sugar alcohol, lactic acid, and the composition may be formulated as described in, e.g., WO 92/19709 and WO 92/19708. The enzymes of the invention may also be stabilized by adding reversible enzyme inhibitors, e.g., of the protein type (as described in EP 0 544 777 B1) or the boronic acid type. Other enzyme stabilizers are well known in the art, such as peptide aldehydes and protein hydrolysate, e.g. the metalloproteases according to the invention may be stabilized using peptide aldehydes or ketones such as described in WO2005/105826 and WO2009/118375.

Protected enzymes for inclusion in a detergent composition of the invention may be prepared, as mentioned above, according to the method disclosed in EP 238 216.

The composition may be augmented with one or more agents for preventing or removing the formation of the biofilm. These agents may include, but are not limited to, dispersants, surfactants, detergents, other enzymes, anti-microbials, and biocides.

### Other Enzymes

In one embodiment, a metalloprotease of the invention is combined with one or more enzymes, such as at least two enzymes, more preferred at least three, four or five enzymes. Preferably, the enzymes have different substrate specificity, e.g., proteolytic activity, amylolytic activity, lipolytic activity, hemicellulytic activity or pectolytic activity.

The detergent additive as well as the detergent composition may comprise one or more enzymes such as a protease, lipase, cutinase, an amylase, carbohydrase, cellulase, pectinase, mannanase, arabinase, galactanase, xylanase, oxidase, e.g., a laccase and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases:

Suitable cellulases include those of animal, vegetable or microbial origin. Particularly suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO 1999/001544.

Commercially available cellulases include Celluzyme™, and Carezyme™ (Novozymes NS), Clazinase™, and Puradax HA™ (Genencor International Inc.), and KAC-500(B)™ (Kao Corporation).

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens. Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the protease variants may comprise the mutations: S3T, V4I, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes NS), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel A G) and KAP (Bacillus alkalophilus subtilisin) from Kao.

### Lipases:

Suitable lipases include those of animal, vegetable or microbial origin. Particularly suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolase™, Lipolase Ultra™, and Lipex™ (Novozymes NS).

### Amylases:

Suitable amylases include those of animal, vegetable or microbial origin. Particularly suitable amylases (alpha and/or beta) include those of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Stainzyme™, Natalase™, Duramyl™, Termamyl™, Fungamyl™ and BAN™ (Novozymes NS), Rapidase™ and Purastar™ (from Genencor International Inc.).

### Peroxidases/Oxidases:

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes NS).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates as described above, liquids, in particular stabilized liquids, or slurries.

### Surfactants

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. In a preferred embodiment the detergent is a liquid or powder detergent comprising less than 40%, preferably less than 30%, more preferably less than 25%, even more preferably less than 20% by weight of surfactant. The composition may comprise from 1% to 15%, preferably from 2% to 12%, 3% to 10%, most preferably from 4% to 8%, even most preferably from 4% to 6% of one or more surfactants. Preferred surfactants are anionic surfactants, non-ionic surfactants, cationic surfactants, zwitterionic surfactants, amphoteric surfactants, and mixtures thereof. Preferably, the major part of the surfactant is anionic. Suitable anionic surfactants are well known in the art and may comprise fatty acid carboxylates (soap), branched-chain, linear-chain and random chain alkyl sulfates or fatty alcohol sulfates or primary alcohol sulfates or alkyl benzenesulfonates such as LAS and LAB or phenylalknesulfonates or alkenyl sulfonates or alkenyl benzenesulfonates or alkyl ethoxysulfates or fatty alcohol ether sulfates or alpha-olefin sulfonate or dodecenyl/tetradecnylsuccinic acid. The anionic surfactants may be alkoxylated. The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant. Suitable non-ionic surfactants are well known in the art and may comprise alcohol ethoxylates, and/or alkyl ethoxylates, and/or alkylphenol ethoxylates, and/or glucamides such as fatty acid N-glucosyl N-methyl amides, and/or alkyl polyglucosides and/or mono- or diethanolamides or fatty acid amides. The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant. Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.5 g/I to 10 g/I detergent composition. Many suitable surface active compounds are available and fully described in the literature, for example, in “Surface-Active Agents and Detergents”, Volumes I and 11, by Schwartz, Perry and Berch.

### Builders

The main role of builder is to sequester divalent metal ions (such as calcium and magnesium ions) from the wash solution that would otherwise interact negatively with the surfactant system. Builders are also effective at removing metal ions and inorganic soils from the fabric surface, leading to improved removal of particulate and beverage stains. Builders are also a source of alkalinity and buffer the pH of the wash water to a level of 9.5 to 11. The buffering capacity is also termed reserve alkalinity, and should preferably be greater than 4.

The detergent compositions of the present invention may comprise one or more detergent builders or builder systems. Many suitable builder systems are described in the literature, for example in Powdered Detergents, Surfactant science series volume 71, Marcel Dekker, Inc. Builder may comprise from 0% to 60%, preferably from 5% to 45%, more preferably from 10% to 40%, most preferably from 15% to 35%, even more preferably from 20% to 30% builder by weight of the subject composition. The composition may comprise from 0% to 15%, preferably from 1% to 12%, 2% to 10%, most preferably from 3% to 8%, even most preferably from 4% to 6% of builder by weight of the subject composition.

Builders include, but are not limited to, the alkali metal, ammonium and alkanolammonium salts of polyphosphates (e.g., tripolyphosphate STPP), alkali metal silicates, alkaline earth and alkali metal carbonates, aluminosilicate builders (e.g., zeolite) and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Ethanole amines (MEA, DEA, and TEA) may also contribute to the buffering capacity in liquid detergents.

### Bleaches

The detergent compositions of the present invention may comprise one or more bleaching agents. In particular powdered detergents may comprise one or more bleaching agents. Suitable bleaching agents include other photobleaches, pre-formed peracids, sources of hydrogen peroxide, bleach activators, hydrogen peroxide, bleach catalysts and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) other photobleaches for example Vitamin K3;

(2) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxone, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;

(3) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include inorganic salts such as alkali metal silicate, carbonate or borate salts or mixtures thereof, or organic materials such as water-soluble or dispersible polymers, waxes, oils or fatty soaps. Useful bleaching compositions are described in U.S. Pat. Nos. 5,576,282, and 6,306,812;

(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof; and

(5) bleach catalysts that are capable of accepting an oxygen atom from peroxyacid and transferring the oxygen atom to an oxidizable substrate are described in WO 2008/007319. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. The bleach catalyst will typically be comprised in the detergent composition at a level of from 0.0005% to 0.2%, from 0.001% to 0.1%, or even from 0.005% to 0.05% by weight.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

### Adjunct Materials

Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino)stilbene-2,2′-disulphonate, 4,4′-bis-(2,4-dianilino-s-triazin-6-ylamino)stilbene-2.2′-disulphonate, 4,4′-bis-(2-anilino-4(N-methyl-N-2-hydroxy-ethylamino)-s-triazin-6-ylamino)stilbene-2,2′-disulphonate, 4,4′-bis-(4-phenyl-2,1,3-triazol-2-yl)stilbene-2,2′-disulphonate, 4,4′-bis-(2-anilino-4(1-methyl-2-hydroxy-ethylamino)-s-triazin-6-ylamino)stilbene-2,2′-disulphonate and, 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino)stilbene disulphonate. Tinopal CBS is the disodium salt of 2,2′-bis-(phenyl-styryl) disulphonate.

Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Fabric Hueing Agents

The detergent compositions of the present invention may also include fabric hueing agents such as dyes or pigments which when formulated in detergent compositions can deposit onto a fabric when said fabric is contacted with a wash liquor comprising said detergent compositions thus altering the tint of said fabric through absorption of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1 876 226. The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series, volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523. Furthermore random graft co-polymers are suitable soil release polymers Suitable graft co-polymers are described in more detail in WO 2007/138054, WO 2006/108856 and WO 2006/113314. Other soil release polymers are substituted polysaccharide structures especially substituted cellulosic structures such as modified cellulose deriviatives such as those described in EP 1 867 808 or WO 2003/040279. Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of: (i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562. Further polymers useful in detergent formulations are described in WO 2007/149806.

In another aspect the detergent is a compact granular (powdered) detergent comprising a) at least about 10%, preferably from 15 to 60% by weight of the composition, of surfactant selected from anionic surfactants, non ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

### Use of the Metalloproteases in Detergents

The soils and stains that are important for detergent formulators are composed of many different substances, and a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. Stain removing enzymes that are known in the art include enzymes such as carbohydrases, amylases, proteases, lipases, cellulases, hemicellulases, xylanases, cutinases, and pectinase.

In one aspect, the present invention concerns the use of metalloproteases of the invention in detergent compositions and cleaning processes, such as laundry and hard surface cleaning. Thus, in one aspect, the present invention demonstrates the detergency effect of the metalloproteases of the invention on various stains and under various conditions. In a particular aspect of the invention the detergent composition and the use in cleaning process concerns the use of a metalloprotease of the invention together with at least one of the above mentioned stain removal enzymes, such as another protease, and in particular a serine protease.

In a preferred aspect of the present invention the metalloproteases useful according to the invention may be combined with at least two enzymes. These additional enzymes are described in details in the section “other enzymes”, more preferred at least three, four or five enzymes. Preferably, the enzymes have different substrate specificity, e.g., carbolytic activity, proteolytic activity, amylolytic activity, lipolytic activity, hemicellulytic activity or pectolytic activity. The enzyme combination may for example be a metalloproteases of the invention with another stain removing enzyme, e.g., a metalloprotease of the invention and a protease, a metalloprotease of the invention and an amylase, a metalloprotease of the invention and a cellulase, a metalloprotease of the invention and a hemicellulase, a metalloprotease of the invention and a lipase, a metalloprotease of the invention and a cutinase, a metalloprotease of the invention and a pectinase or a metalloprotease of the invention and an anti-redeposition enzyme. More preferably, the metalloproteases of the invention is combined with at least two other stain removing enzymes, e.g., a metalloprotease of the invention, a lipase and an amylase; or a metalloprotease of the invention, a protease and an amylase; or a metalloprotease of the invention, a protease and a lipase; or a metalloprotease of the invention, a protease and a pectinase; or a metalloprotease of the invention, a protease and a cellulase; or a metalloprotease of the invention, a protease and a hemicellulase; or a metalloprotease of the invention, a protease and a cutinase; or a metalloprotease of the invention, an amylase and a pectinase; or a metalloprotease of the invention, an amylase and a cutinase; or a metalloprotease of the invention, an amylase and a cellulase; or a metalloprotease of the invention, an amylase and a hemicellulase; or a metalloprotease of the invention, a lipase and a pectinase; or a metalloprotease of the invention, a lipase and a cutinase; or a metalloprotease of the invention, a lipase and a cellulase; or a metalloprotease of the invention, a lipase and a hemicellulase. Even more preferably, the metalloproteases of the invention may be combined with at least three other stain removing enzymes, e.g., a metalloprotease of the invention, a protease, a lipase and an amylase; or a metalloprotease of the invention, a protease, an amylase and a pectinase; or a metalloprotease of the invention, a protease, an amylase and a cutinase; or a metalloprotease of the invention, a protease, an amylase and a cellulase; or a metalloprotease of the invention, a protease, an amylase and a hemicellulase; or a metalloprotease of the invention, an amylase, a lipase and a pectinase; or a metalloprotease of the invention, an amylase, a lipase and a cutinase; or a metalloprotease of the invention, an amylase, a lipase and a cellulase; or a metalloprotease of the invention, an amylase, a lipase and a hemicellulase; or a metalloprotease of the invention, a protease, a lipase and a pectinase; or a metalloprotease of the invention, a protease, a lipase and a cutinase; or a metalloprotease of the invention, a protease, a lipase and a cellulase; or a metalloprotease of the invention, a protease, a lipase and a hemicellulase. A metalloprotease according to the present invention may be combined with any of the enzymes selected from the non-exhaustive list comprising: carbohydrases, such as an amylase, a hemicellulase, a pectinase, a cellulase, a xanthanase or a pullulanase, a peptidase, a protease or a lipase. In a preferred embodiment, a metalloprotease of the invention is combined with a serine protease, e.g., an S8 family protease such as Savinase®.

In another embodiment of the present invention, a metalloprotease of the invention useful according to the present invention may be combined with one or more other metalloproteases, such as another M4 Metalloprotease, including Neutrase® or Thermolysin. Such combinations may further comprise combinations of the other detergent enzymes as outlined above.

The cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one metalloprotease of the invention. The cleaning process or a textile care process can for example be carried out in a machine washing process or in a manual washing process. The washing solution can for example be an aqueous washing solution containing a detergent composition.

The fabrics and/or garments subjected to a washing, cleaning or textile care process of the present invention may be conventional washable laundry, for example household laundry. Preferably, the major part of the laundry is garments and fabrics, including knits, woven, denims, non-woven, felts, yarns, and to welling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

The invention further concerns the use of metalloproteases of the invention in a proteinaceous stain removing processes. The proteinaceous stains may be stains such as food stains, e.g., baby food, sebum, cocoa, egg, blood, milk, ink, grass, or a combination hereof.

Typical detergent compositions includes various components in addition to the enzymes, these components have different effects, some components like the surfactants lower the surface tension in the detergent, which allows the stain being cleaned to be lifted and dispersed and then washed away, other components like bleach systems removes discolor often by oxidation and many bleaches also have strong bactericidal properties, and are used for disinfecting and sterilizing. Yet other components like builder and chelator softens, e.g., the wash water by removing the metal ions form the liquid.

In a particular embodiment, the invention concerns the use of a composition comprising a metalloprotease of the invention, wherein said enzyme composition further comprises at least one or more of the following a surfactant, a builder, a chelator or chelating agent, bleach system or bleach component in laundry or dish wash.

In a preferred embodiment of the invention the amount of a surfactant, a builder, a chelator or chelating agent, bleach system and/or bleach component are reduced compared to amount of surfactant, builder, chelator or chelating agent, bleach system and/or bleach component used without the added metalloprotease of the invention. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of metalloprotease of the invention, such as a conventional amount of such component. In one aspect, the metalloprotease of the invention is used in detergent compositions wherein said composition is free of at least one component which is a surfactant, a builder, a chelator or chelating agent, bleach system or bleach component and/or polymer.

### Washing Method

The detergent compositions of the present invention are ideally suited for use in laundry applications. Accordingly, the present invention includes a method for laundering a fabric. The method comprises the steps of contacting a fabric to be laundered with a cleaning laundry solution comprising the detergent composition according to the invention. The fabric may comprise any fabric capable of being laundered in normal consumer use conditions. The solution preferably has a pH of from about 5.5 to about 8. The compositions may be employed at concentrations of from about 100 ppm, preferably 500 ppm to about 15,000 ppm in solution. The water temperatures typically range from about 5° C. to about 90° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8.

In particular embodiments, the washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

The present invention relates to a method of cleaning a fabric, a dishware or hard surface with a detergent composition comprising a metalloprotease of the invention.

A preferred embodiment concerns a method of cleaning, said method comprising the steps of: contacting an object with a cleaning composition comprising a metalloprotease of the invention under conditions suitable for cleaning said object. In a preferred embodiment the cleaning composition is a detergent composition and the process is a laundry or a dish wash process.

Still another embodiment relates to a method for removing stains from fabric which comprises contacting said a fabric with a composition comprising a metalloprotease of the invention under conditions suitable for cleaning said object.

In a preferred embodiment the compositions for use in the methods above further comprises at least one additional enzyme as set forth in the “other enzymes” section above, such as an enzyme selected from the group consisting of carbohydrases, peptidases, proteases, lipases, cellulase, xylanases or cutinases or a combination hereof. In yet another preferred embodiment the compositions comprises a reduced amount of at least one or more of the following components a surfactant, a builder, a chelator or chelating agent, bleach system or bleach component or a polymer.

Also contemplated are compositions and methods of treating fabrics (e.g., to desize a textile) using one or more of the metalloprotease of the invention. The metalloproteases can be used in any fabric-treating method which is well known in the art (see, e.g., U.S. Pat. No. 6,077,316). For example, in one aspect, the feel and appearance of a fabric is improved by a method comprising contacting the fabric with a metalloprotease in a solution. In one aspect, the fabric is treated with the solution under pressure.

### Low Temperature Uses

One embodiment of the invention concerns a method of doing laundry, dish wash or industrial cleaning comprising contacting a surface to be cleaned with the metalloproteases of the invention, and wherein said laundry, dish wash, industrial or institutional cleaning is performed at a temperature of about 40° C. or below. One embodiment of the invention relates to the use of a metalloprotease in laundry, dish wash or a cleaning process wherein the temperature in laundry, dish wash, industrial cleaning is about 40° C. or below

In another embodiment, the invention concerns the use of a metalloprotease according to the invention in a protein removing process, wherein the temperature in the protein removing process is about 40° C. or below.

The present invention also relates to the use in laundry, dish wash or industrial cleaning process of a metalloprotease having at least one improved property compared to a commercial metalloprotease such as Neutrase® and wherein the temperature in laundry, dish wash or cleaning process is performed at a temperature of about 40° C. or below.

In each of the above-identified methods and uses, the wash temperature is about 40° C. or below, such as about 39° C. or below, such as about 38° C. or below, such as about 37° C. or below, such as about 36° C. or below, such as about 35° C. or below, such as about 34° C. or below, such as about 33° C. or below, such as about 32° C. or below, such as about 31° C. or below, such as about 30° C. or below, such as about 29° C. or below, such as about 28° C. or below, such as about 27° C. or below, such as about 26° C. or below, such as about 25° C. or below, such as about 24° C. or below, such as about 23° C. or below, such as about 22° C. or below, such as about 21° C. or below, such as about 20° C. or below, such as about 19° C. or below, such as about 18° C. or below, such as about 17° C. or below, such as about 16° C. or below, such as about 15° C. or below, such as about 14° C. or below, such as about 13° C. or below, such as about 12° C. or below, such as about 11° C. or below, such as about 10° C. or below, such as about 9° C. or below, such as about 8° C. or below, such as about 7° C. or below, such as about 6° C. or below, such as about 5° C. or below, such as about 4° C. or below, such as about 3° C. or below, such as about 2° C. or below, such as about 1° C. or below.

In another preferred embodiment, the wash temperature is in the range of about 5-40° C., such as about 5-30° C., about 5-20° C., about 5-10° C., about 10-40° C., about 10-30° C., about 10-20° C., about 15-40° C., about 15-30° C., about 15-20° C., about 20-40° C., about 20-30° C., about 25-40° C., about 25-30° C., or about 30-40° C. In a particular preferred embodiment the wash temperature is about 30° C.

In particular embodiments, the low temperature washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8.

In particular embodiments, the low temperature washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

### Use in Removing Egg Stains

Another particular embodiment of the invention concerns removal of egg stains. These types of stain are often very difficult to remove completely. Egg stains are particularly problematic in hard surface cleaning such as dish wash where the stains often remain on the plates and cutlery after washing. The metalloproteases of the invention are particularly suitable for removing egg stains.

Thus, the invention further concerns methods for removing egg stains from textiles, fabrics and/or hard surfaces like dishes and cutlery in particular from fabrics and textiles. A preferred aspect of the invention concerns a method of removing egg stains from textiles and/or fabrics comprising contacting a surface in need of removal of an egg stain with a metalloprotease of the invention. In one embodiment, the invention comprises a method of removing egg stains from textiles and/or fabrics comprising contacting a surface in need of removal of an egg stain with a detergent composition comprising a metalloprotease of the invention. The invention also concerns a method of removing egg stains comprising adding a metalloprotease of the invention to a laundry and/or washing process wherein said textiles and/or fabric comprises various egg stains.

One embodiment of the present invention relates to a method for removal of egg stains from a hard surface or from laundry, the method comprising contacting the egg stain-containing hard surface or the egg stain-containing laundry with a cleaning or detergent composition, preferably a laundry or dish wash composition, containing a metalloprotease of the invention.

Another embodiment relates a method for removing egg stains from fabric or textile which comprises contacting the fabric or textile with a cleaning or detergent composition, preferably a laundry or dish wash composition, comprising a metalloprotease of the invention.

A still further embodiment relates to a method for removing egg stains from fabric or textile which comprises contacting said a fabric or textile with a composition comprising a metalloprotease of the invention, wherein said composition further comprises at least one additional enzyme as set forth in the “other enzymes” section above, such as an enzyme selected from the group consisting of a carbohydrase, a peptidase, a protease, a lipase, a cellulase, a xylanase, a cutinase or a combination hereof.

In particular embodiments, the egg removing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8.

In particular embodiments, the egg removing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

All documents cited herein are incorporated by reference in the entirety.

The present invention is further described by the following examples that should not be construed as limiting the scope of the invention.

### EXAMPLES

### Materials and Methods

### Wash Assays

### Automatic Mechanical Stress Assay (AMSA) for Laundry

In order to assess the wash performance in laundry washing experiments are performed, using the Automatic Mechanical Stress Assay (AMSA). With the AMSA, the wash performance of a large quantity of small volume enzyme-detergent solutions can be examined. The AMSA plate has a number of slots for test solutions and a lid firmly squeezing the laundry sample, the textile to be washed against all the slot openings. During the washing time, the plate, test solutions, textile and lid are vigorously shaken to bring the test solution in contact with the textile and apply mechanical stress in a regular, periodic oscillating manner. For further description see WO02/42740 especially the paragraph “Special method embodiments” at page 23-24.

The wash performance is measured as the brightness of the colour of the textile washed. Brightness can also be expressed as the intensity of the light reflected from the sample when illuminated with white light. When the sample is stained the intensity of the reflected light is lower, than that of a clean sample. Therefore the intensity of the reflected light can be used to measure wash performance.

Colour measurements are made with a professional flatbed scanner (Kodak iQsmart, Kodak, Midtager 29, DK-2605 Brøndby, Denmark), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

TABLE 1
Composition of model detergents and test materials

| | |
|---|---|
| Laundry powder | Sodium citrate dihydrate 32.3% |
| model detergent A | Sodium-LAS 24.2% |
| | Sodium lauryl sulfate 32.2% |
| | Neodol 25-7 (alcohol ethoxylate) 6.4% |
| | Sodium sulfate 4.9% |
| Laundry liquid | Water 30.63% |
| model detergent B | Sodium hydroxide 2.95% |
| | Dodecylbenzensulfonic acid 11.52% |
| | Fatty acids (Soya) 5.50% |
| | Propane-1,2-diol (MPG) 5.05% |
| | Water 17.38% |
| | C13-alcohol ethoxylate, 10.50% |
| | Diethylenetriaminepentakis (methylenephosphonic |
| | acid) (DTMPA) 3.08% |
| | Triethanolamine (TEA) 2.22% |
| | Fatty acids (Coco) 4.50% |
| | Sodium citrate monohydrate 1.00% |
| | Ethanol 4.63% |
| | Syntran 5909 (opacifier) 0.30% |
| | Perfume 0.35% |
| Test material | PC-03 (Chocolate-milk/ink on cotton/polyester) |
| | C-10 (Oil/milk/pigment on cotton) |
| | PC-05 (Blood/milk/ink on cotton/polyester) |
| | EMPA117EH (Blood/milk/ink on cotton/polyester) |

Test materials are obtained from Center For Testmaterials BV, P.O. Box 120, 3133 KT Vlaardingen, the Netherlands and EMPA Testmaterials AG, Movenstrasse 12, CH-9015 St. Gallen, Switzerland.

### Protease Assays

### Protazyme AK Purification Activity Assay:

Substrate: Protazyme AK tablet (AZCL-casein, Megazyme T-PRAK 1000).

### Temperature: 37° C.

Assay buffer: 50 mM HEPES/NaOH, pH 7.0.

A Protazyme AK tablet is suspended in 2.0 ml 0.01% Triton X-100 by gentle stirring. 500 μl of this suspension and 500 μl assay buffer are dispensed in an Eppendorf tube and placed on ice. 20 μl protease sample (diluted in 0.01% Triton X-100) is added to the ice cold tube. The assay is initiated by transferring the Eppendorf tube to an Eppendorf thermomixer, which is set to the assay temperature. The tube is incubated for 15 minutes on the Eppendorf thermomixer at its highest shaking rate (1400 rpm). The incubation is stopped by transferring the tube back to the ice bath. Then the tube is centrifuged in an ice cold centrifuge for a few minutes and 200 μl supernatant is transferred to a microtiter plate. OD₆₅₀ is read as a measure of protease activity. A buffer blind is included in the assay (instead of enzyme).

### Characterization Activity Assays:

### Protazyme OL Characterization Assay:

Substrate: Protazyme OL tablet (AZCL-collagen, Megazyme T-PROL 1000).
Temperature: Controlled (assay temperature).
Assay buffers: 100 mM succinic acid, 100 mM HEPES, 100 mM CHES, 100 mM CABS, 1 mM CaCl₂, 150 mM KCl, 0.01% Triton X-100 adjusted to pH-values 2.0, 3.0, 4.0, 5.0, 6.0, 7.0, 8.0, 9.0, 10.0 and 11.0 with HCl or NaOH.

A Protazyme OL tablet is suspended in 2.0 ml 0.01% Triton X-100 by gentle stirring. 500 μl of this suspension and 500 μl assay buffer are dispensed in an Eppendorf tube and placed on ice. 20 μl protease sample (diluted in 0.01% Triton X-100) is added to the ice cold tube. The assay is initiated by transferring the Eppendorf tube to an Eppendorf thermomixer, which is set to the assay temperature. The tube is incubated for 15 minutes on the Eppendorf thermomixer at its highest shaking rate (1400 rpm). The incubation is stopped by transferring the tube back to the ice bath. Then the tube is centrifuged in an ice cold centrifuge for a few minutes and 200 μl supernatant is transferred to a microtiter plate. OD₆₅₀ is read as a measure of protease activity. A buffer blind is included in the assay (instead of enzyme).

### Example 1

### Design of Optimized DNA Sequence

The sequence of two family M4 proteases derived from strains belonging to the genus Exiguobacterium were identified in the public protein sequence database SWISSPROT having the accession number SWISSPROT:BIYFRI (Exiguobacterium sibiricum) (SEQ ID NO: 1) and SWISSPROT: C4LIB3 (Exiguobacterium sp.AT1b) (SEQ ID NO: 3). In order to express the metalloproteases, genes encoding the metalloproteases were codon usage optimized for expression in a Bacillus subtilis host strain, resulting in sequences shown in SEQ ID NO: 5 and SEQ ID NO: 6. The codon optimization process is a method known in the art and is also described in WO 2012025577.

Expression of the Metalloproteases Exiguobacterium sibiricum

A DNA fragment having the sequence of SEQ ID NO: 1 (Exiguobacterium sibiricum) was codon optimized and synthesized as described above in this example and cloned as essentially described in WO 2010151787. The signal peptide from the alkaline protease from B. clausii (aprH) was fused by PCR as described in WO 99/43835 in frame to the DNA fragment encoding the metalloprotease from Exiguobacterium sibiricum using the synthetic DNA fragment as template and the oligos.

forward primer:
(SEQ ID NO 7)
GTTCATCGATCGCATCGGCTGAGGGCCTTCAAGCTGGC
reverse primer:
(SEQ ID NO 8)
GCGTTTTTTTATTGATTAACGCGTTTAGTATACGCCAACTGCG

The nucleotide fragment obtained from the subsequent PCR was integrated by homologous recombination into the Bacillus subtilis host cell genome as described in WO2010/151787. Selection of positive clones on LB medium agar plates which contained 6 ug/mL chloramphenicol and 1% skimmed milk and cultivation of clones in liquid rich medium was done as described in WO2010/151787. The expression clone was cultivated for 48 hours at 26° C. in a protein rich bacterial growth medium prior to protein purification. The supernatant was separated from the B. subtilis cells by centrifugation at 10000 rpm for 30 minutes followed by filtration through a 0.45 μm filter. The supernatant was stored at −20° C. until further use. Successful expression was detected by SDS-PAGE analysis showing a protein band at approximately 33 kDa above host background.

Expression of the Metalloproteases Exiguobacterium sp. AT1b

A DNA fragment having the sequence of SEQ ID NO: 3 (Exiguobacterium sp. AT1b) was synthesized (Geneart®, Life Technologies, Naerum, Denmark) and the synthetic gene was cloned into the Bacillus subtilis expression vector according to the procedure essentially described in WO 2012025577 and WO 2010151787. The expression vector construct denoted C362H encoded a fusion protein in which the natural signal peptide has been replaced by the aprH signal peptide from B. clausii as described in WO 99/43835. E. coli TOP10 cells were transformed with plasmid C362H and one correct clone was selected on media containing ampicillin by using methods known in the art. The nucleotide fragment encoding the metalloprotease fusion coding sequence was integrated by homologous recombination into the Bacillus subtilis host cell genome. The gene construct was expressed under the control of a triple promoter system (as described in WO 99/43835). The gene coding for chloramphenicol acetyltransferase was used as marker (as described in (Diderichsen et al., 1993, Plasmid 30: 312-315).

Ten B. subtilis clones were separated on LB medium agar plates which contained 6 ug/mL chloramphenicol and 1% skimmed milk. The agar plates were incubated over night at 37° C. and clones expressing the polypeptide having protease activity showed clearing zones around the colonies indicating the presence of proteolytic activity.

One clone expressing the metalloprotease was grown in rich liquid medium at 37° C. and shaking 225 rpm for 4 to 5 days. The supernatant was separated from the B. subtilis cells by centrifugation at 10000 rpm for 30 minutes followed by filtration through a 0.45 μm filter. The supernatant was stored at −20° C. until further use. 40 μl thawed sample was added 10 μl 50% trichloro acetic acid (TCA) and incubated on ice for 5 minutes before centrifugation 40000 rpm for 5 minutes. The supernatant was removed and 20 μl SDS-PAGE sample buffer (Nupage, Invitrogen) was added to the pellet followed by pipetting up and down until the pellet was completely dissolved. A clear protein band of approximately 34 kDa was detected above the host background, indicating successful recombinant expression of the metalloprotease.

### Example 2

### Purification of the M4 Protease from Exiguobacterium Sibiricum

(The M4 Protease was Expressed in B. subtilis.)

The culture broth was centrifuged (20000×g, 20 min) and the supernatant was carefully decanted from the precipitate. The supernatant was filtered through a Nalgene 0.2 μm filtration unit in order to remove the rest of the Bacillus host cells. The 0.2 μm filtrate was applied to a Bacitracin agarose column (from Upfront chromatography) equilibrated in 20 mM MES/NaOH, 5 mM CaCl₂, pH 6. After washing the column extensively with the equilibration buffer, the M4 protease was eluted with 100 mM H₃BO₃, 10 mM MES, 2 mM CaCl₂, 1M NaCl, pH 6 with 25% (v/v) 2-propanol. Fractions from the column were analyzed for protease activity (Protazyme AK purification activity assay at pH 7) and active fractions were further analyzed by SDS-PAGE. Fractions, where only one band was seen on the coomassie stained SDS-PAGE gel, were pooled and transferred to 100 mM H₃BO₃, 10 mM MES, 2 mM CaCl₂, pH 6 on a G25 sephadex column as the purified preparation and was used for further characterization.

Purification of the M4 Protease from Exiguobacterium sp. AT1b

(The M4 Protease was Expressed in B. subtilis.)

The culture broth was centrifuged (20000×g, 20 min) and the supernatant was carefully decanted from the precipitate. The supernatant was filtered through a Nalgene 0.2 μm filtration unit in order to remove the rest of the Bacillus host cells. The 0.2 μm filtrate was diluted 2.5-fold in deionized water and applied to a Bacitracin agarose column (from Upfront chromatography) equilibrated in 20 mM MES/NaOH, 5 mM CaCl₂, pH 6. After washing the column extensively with the equilibration buffer, the M4 protease was eluted with 100 mM H₃BO₃, 10 mM MES, 2 mM CaCl₂, 1M NaCl, pH 6 with 25% (v/v) 2-propanol. Fractions from the column were analyzed for protease activity (Protazyme AK purification activity assay at pH 7) and active fractions were further analyzed by SDS-PAGE. Fractions, where only one band was seen on the coomassie stained SDS-PAGE gel, were pooled and transferred to 100 mM H₃BO₃, 10 mM MES, 2 mM CaCl₂, pH 6 on a G25 sephadex column as the purified preparation and was used for further characterization.

### Example 3

### Characterization of the M4 Proteases from Exiguobacterium: pH-Activity, pH-Stability, and Temperature Activity

The Protazyme OL characterization assay was used for obtaining the pH-activity profile at 37° C., the pH-stability profile (residual activity after 2 hours at indicated pH-values) and the temperature-activity profile at pH optimum. For the pH-stability profile the protease was diluted 7× in the different characterization assay buffers to reach the pH-values of these buffers and incubated for 2 hours at 37° C. After incubation, the pH of the protease incubations was transferred to the pH optimum of the protease, before assay for residual activity, by dilution in the pH optimum assay buffer. The results are shown in Tables 2-4 below. For Table 2, the activities are relative to the optimal pH for the enzyme. For Table 3, the activities are residual activities relative to a sample, which was kept at stable conditions (5° C., pH 6). For Table 4, the activities are relative to the optimal temperature at pH optimum for the enzyme.

TABLE 2
pH-activity profile at 37° C.

| | M4 protease from | M4 protease from | M4 protease from |
|---|---|---|---|
| | Exiguobacterium | Exiguobacterium | Bacillus |
| pH | sibiricum | sp. AT1b | amyloliquefaciens |
| 2 | 0.01 | 0.00 | 0.00 |
| 3 | 0.00 | 0.00 | 0.00 |
| 4 | 0.00 | 0.00 | 0.00 |
| 5 | 0.06 | 0.08 | 0.39 |
| 6 | 0.75 | 0.58 | 1.00 |
| 7 | 0.76 | 0.63 | 0.98 |
| 8 | 1.00 | 0.93 | 0.59 |
| 9 | 0.47 | 1.00 | 0.16 |
| 10 | 0.01 | 0.09 | 0.01 |
| 11 | 0.03 | 0.00 | 0.00 |

TABLE 3
pH-stability profile (residual activity after 2 hours at 37° C.)

| | M4 protease from | M4 protease from | M4 protease from |
|---|---|---|---|
| | Exiguobacterium | Exiguobacterium sp. | Bacillus |
| pH | sibiricum | AT1b | amyloliquefaciens |
| 2 | — | 0.00 | 0.00 |
| 3 | 0.00 | 0.00 | 0.00 |
| 4 | 0.00 | 0.01 | 0.00 |
| 5 | 0.00 | 0.00 | 0.98 |
| 6 | 0.38 | 0.88 | 1.00 |
| 7 | 0.31 | 0.91 | 1.01 |
| 8 | 0.07 | 0.75 | 0.99 |
| 9 | 0.02 | 0.46 | 0.95 |
| 10 | 0.01 | 0.01 | 0.87 |
| 11 | — | 0.01 | 0.17 |
| After 2 | 1.00 | 1.00 | 1.00 |
| hours at | (at pH 6) | (at pH 6) | (at pH 6) |
| 5° C. | | | |

TABLE 4
Temperature activity profile at pH 6, pH 8 or pH 9

| | M4 protease from | M4 protease from | M4 protease from |
|---|---|---|---|
| Temp | Exiguobacterium | Exiguobacterium sp. | Bacillus |
| (° C.) | sibiricum | AT1b | amyloliquefaciens |
| 15 | 0.22 | 0.10 | 0.07 |
| 25 | 0.37 | 0.29 | 0.20 |
| 37 | 1.00 | 1.00 | 0.80 |
| 50 | 0.32 | 0.93 | 1.00 |

Other Characteristics for the M4 Proteases from Exiguobacterium

### The M4 Proteases are Inhibited by 1,10-Phenanthroline and EDTA.

Exiguobacterium sibiricum

Determination of the N-terminal sequence by EDMAN degradation was: VTGTTSV (SEQ ID NO 11). The relative molecular weight as determined by SDS-PAGE was approx. Mᵣ=36 kDa. The molecular weight determined by intact molecular weight analysis was 33543.5 Da. The mature sequence (from mass spectrometry data and EDMAN degradation data and DNA sequence):

(SEQ ID NO 9)
VTGTTSVGTGTTVLGTTATFNTVKSGSYYYLQDSTRGKGIYTYDAKK
RNTLPGSLWADLDNQFNTTYDRAAVSAQVNAVKTYDFYKNTYGRNSY
DNAGAALNSSVHYSTSYNNAFWDGTKMVYGDGDGSTFTYLSGALDVV
AHELTHAVTEYTAGLVYQNESGAINEAVSDIMGTVAEYSVGSNFDWL
VGEDIYTPGVSGDALRSMSNPAAYGDPDHYSKRYTGTQDNGGVHTNS
GIVNKAAYLLGNGGTHTGVTVTGVGVPKLGAIYYRALSVYLTPNSNF
SSLRAAVVQSAKDLYGSTSAEATAAAKSFDAVGVY

Amino acids 1 to 317 of SEQ ID NO: 2.

The calculated molecular weight from this mature sequence was 33543.5 Da.

### Exiguobacterium sp.AT1b.

Determination of the N-terminal sequence by EDMAN degradation was: GTTYTGT (SEQ ID NO 12)

The relative molecular weight as determined by SDS-PAGE was approx. Mᵣ=36 kDa.

The molecular weight determined by intact molecular weight analysis was 33770.9 Da. The mature sequence (from mass spectrometry data and EDMAN degradation data and DNA sequence):

(SEQ ID NO 10)
GTTYTGTGIDVLGYSQTFKTTKSGSYYYLQDSTRGKGIYTYDAKNRT
TLPGSLWADVDNVLNTTYDRAAVSAHVNATKTYDFYKNTYGRNSYDN
AGAALNSTVHYSRSYNNAFWDGSKMVYGDGDGQTFTYLSGALDVVAH
ELTHAITEYTAGLIYQNESGAINEAVSDILGTVAEYSVGTNFDWLVG
EDIYTPGVAGDGLRSMANPAAYGDPDHYSKRYTGTQDNGGVHINSGI
VNKAAYLLGNGGSHYGVSVQGVGVMAMGDIYYRALNVYLTPTSNFSS
LRQAVVQSAKDLYGATSPQAVSAAKSFDAVGIY

Amino acids 1 to 315 of SEQ ID NO: 4.

The calculated molecular weight from this mature sequence was 33769.8 Da

### Example 4

### AMSA Wash Performance of M4 Proteases from Exiguobacterium

The wash performance of M4 proteases from Exiguobacterium was tested using a model liquid detergent, a commercial liquid detergent and a powder detergent at 2 different wash temperatures on 3 different technical stains using the Automatic Mechanical Stress Assay.

The experiments were conducted as described in the AMSA for laundry method using a single cycle wash procedure described in table 5, with the detergent composition and swatches and the experimental conditions as specified in table 6 and 7 below.

TABLE 5
Experimental conditions for AMSA for tables 6 and 7

| | |
|---|---|
| Test solution | 2.5 g/L powder model detergent A, 2 g/L or |
| | 8 g/L liquid model detergent B and |
| | Small&Mighty 1.33 g/L |
| Test solution volume | 160 micro L |
| pH | As is |
| Wash time | 20 minutes |
| Temperature | 20° C. or 40° C. |
| Water hardness | 15°dH |
| Protease | 0 (blank) or 30 nM |
| concentration | PC-05, Blood/milk/ink on cotton/polyester |
| Swatch | PC-03, Chocolate-milk/soot on cotton/Polyester |
| | CS-37, Full egg with pigment on Cotton |

Water hardness was adjusted to 15° dH by addition of CaCl₂, MgCl₂, and NaHCO₃ (Ca²⁺:Mg²⁺:CO₃²⁻=4:1:7.5) to the test system. After washing the textiles were flushed in tap water and dried.

Table 6: Delta Intensity Value of Detergent Containing M4 Proteases from Exiguobacterium Compared to Detergent without Protease

TABLE 6
Exiguobacterium sibiricum

| | | | | Small & | | | | Small & |
|---|---|---|---|---|---|---|---|---|
| | | | | Mighty | | | | Mighty |
| | Detergent A | Detergent B | Detergent B | (1.33 g/L) | Detergent A | Detergent B | Detergent B | (1.33 g/L) |
| | (2.5 g/L) | (2 g/L) at | (8 g/L) at | at | (2.5 g/L) | (2 g/L) at | (8 g/L) at | at |
| Swatch | at 20° C. | 20° C. | 20° C. | 20° C. | at 40° C. | 40° C. | 40° C. | 40° C. |
| PC-05 | 55 | 35 | 46 | 40 | 69 | 51 | 70 | 63 |
| PC-03 | 19 | 11 | 17 | 10 | 22 | 11 | 35 | 12 |
| CS-37 | 17 | 59 | 23 | 56 | 25 | 63 | 27 | 69 |

TABLE 7
Exiguobacterium sp. AT1b

| | | | | Small & | | | | Small & |
|---|---|---|---|---|---|---|---|---|
| | | | | Mighty | | | | Mighty |
| | Detergent A | Detergent B | Detergent B | (1.33 g/L) | Detergent A | Detergent B | Detergent B | (1.33 g/L) |
| | (2.5 g/L) | (2 g/L) at | (8 g/L) at | at | (2.5 g/L) | (2 g/L) at | (8 g/L) at | at |
| Swatch | at 20° C. | 20° C. | 20° C. | 20° C. | at 40° C. | 40° C. | 40° C. | 40° C. |
| PC-05 | 61 | 35 | 46 | 40 | 78 | 53 | 70 | 64 |
| PC-03 | 24 | 11 | 20 | 12 | 38 | 13 | 40 | 11 |
| CS-37 | 20 | 51 | 22 | 52 | 27 | 66 | 31 | 74 |

The results show that both the Exiguobacterium sibiricum metalloprotease and the Exiguobacterium sp. AT1b metalloprotease show good wash performance on blood/milk/ink (PC-05), chocolate milk/soot (C-03) and full egg stains (CS-37).

Table 8 and 9: Relative Wash Performance Value of Detergent Containing M4 Proteases from Exiguobacterium Compared to Compared to Detergent Containing Neutrase® (SEQ ID NO 13)

TABLE 8
Exiguobacterium sibiricum

| | | | | Small & | | | | Small & |
|---|---|---|---|---|---|---|---|---|
| | | | | Mighty | | | | Mighty |
| | Detergent A | Detergent B | Detergent B | (1.33 g/L) | Detergent A | Detergent B | Detergent B | (1.33 g/L) |
| | (2.5 g/L) | (2 g/L) at | (8 g/L) at | at | (2.5 g/L) | (2 g/L) at | (8 g/L) at | at |
| Swatch | at 20° C. | 20° C. | 20° C. | 20° C. | at 40° C. | 40° C. | 40° C. | 40° C. |
| PC-05 | 0.7 | 0.9 | 0.7 | 1.0 | 0.7 | 1.5 | 0.8 | 1.4 |
| PC-03 | 0.5 | 1.1 | 0.5 | 0.8 | 0.4 | 1.0 | 0.6 | 1.2 |
| CS-37 | 3.0 | 5.5 | 3.1 | 3.7 | 5.6 | 5.6 | 4.9 | 5.5 |

The results show that Exiguobacterium sibiricum metalloprotease surpasses the performance of Neutrase® on the egg stain CS-37 whilst it shows similar or slightly improved wash performance to Neutrase® on the other stains under the tested conditions.

TABLE 9
Exiguobacterium sp. AT1b

| | | | | Small & | | | | Small & |
|---|---|---|---|---|---|---|---|---|
| | | | | Mighty | | | | Mighty |
| | Detergent A | Detergent B | Detergent B | (1.33 g/L) | Detergent A | Detergent B | Detergent B | (1.33 g/L) |
| | (2.5 g/L) | (2 g/L) at | (8 g/L) at | at | (2.5 g/L) | (2 g/L) at | (8 g/L) at | at |
| Swatch | at 20° C. | 20° C. | 20° C. | 20° C. | at 40° C. | 40° C. | 40° C. | 40° C. |
| PC-05 | 0.7 | 0.9 | 0.7 | 1.0 | 0.8 | 1.5 | 0.8 | 1.4 |
| PC-03 | 0.6 | 1.1 | 0.5 | 1.0 | 0.6 | 1.3 | 0.7 | 1.1 |
| CS-37 | 3.4 | 4.8 | 3.1 | 3.4 | 6.1 | 5.7 | 5.8 | 5.9 |

The results show that Exiguobacterium sp. 1b metalloprotease surpasses the performance of Neutrase® on the egg stain CS-37 whilst it shows similar or slightly improved wash performance to Neutrase® on the other stains under the tested conditions.

### Example 5

### AMSA Wash Performance of M4 Protease from Exiguobacterium sibiricum Compared to Savinase®

The wash performance of the M4 protease from Exiguobacterium sibiricum was tested using a liquid model detergent and a commercial liquid detergent at 2 different wash temperatures on 2 different technical stains using the Automatic Mechanical Stress Assay.

The experiments were conducted as described in the AMSA for laundry method using a single cycle wash procedure described in table 10, with the detergent composition and swatches and the experimental conditions as specified in table 11 below.

TABLE 10
Experimental conditions for AMSA for table 11

| | |
|---|---|
| Test solution | 2 g/L or 8 g/L liquid model detergent B and |
| | 1.33 g/L Unilever Persil Small & Mighty |
| Test solution volume | 160 micro L |
| pH | As is |
| Wash time | 20 minutes |
| Temperature | 20° C. or 40° C. |
| Water hardness | 15°dH |
| Protease concentration | 0 (blank) or 30 nM |
| Swatch | PC-03 (Chocolate/milk/soot), PC-05 |
| | (Blood/milk/ink) |

Water hardness was adjusted to 15° dH by addition of CaCl₂, MgCl₂, and NaHCO₃ (Ca²⁺:Mg²⁺:CO₃²⁻=4:1:7.5) to the test system. After washing the textiles were flushed in tap water and dried.

### Table 11: Relative Wash Performance Value of Detergent Containing the M4 Exiguobacterium Sibiricum Proteases Compared to Detergent Containing Savinase® (SEQ ID NO 14)

TABLE 11
Exiguobacterium sibiricum

| | | | Small & | | | Small & |
|---|---|---|---|---|---|---|
| | Detergent B | Detergent B | Mighty | Detergent B | Detergent B | Mighty |
| | (2 g/L) at | (8 g/L) at | (1.33 g/L) | (2 g/L) at | (8 g/L) at | (1.33 g/L) |
| Swatch | 20° C. | 20° C. | at 20° C. | 40° C. | 40° C. | at 40° C. |
| PC-03 | 1.1 | 1.5 | 0.7 | 0.9 | 1.1 | 0.8 |
| PC-05 | 1.6 | 1.3 | 1.3 | 1.1 | 1.2 | 1.3 |

M4 protease Exiguobacterium sibiricum performs generally better than Savinase® on PC-05 and comparable to Savinase® on PC-03 under the investigated conditions.

What is claimed is:

1-9. (canceled)

10. A detergent composition comprising a polypeptide having protease activity wherein the polypeptide having protease activity is selected from the group consisting of:
a) a polypeptide having at least 80% identity to the mature polypeptide of SEQ ID NO: 2 or SEQ ID NO 4;
(b) a polypeptide encoded by a polynucleotide having at least 80% identity to the mature polypeptide coding sequence of SEQ ID NO: 1 or SEQ ID NO 3;
(c) a variant comprising a substitution, deletion, and/or insertion of one or more (several) amino acids of the mature polypeptide of SEQ ID NO: 2 or SEQ ID NO 3; and
(d) a fragment of a polypeptide of (a), (b) or (c) that has protease activity and a surfactant.

11. The detergent composition of claim 10 further comprising one or more additional enzymes selected from the group consisting of a protease, lipase, cutinase, an amylase, carbohydrase, cellulase, pectinase, mannanase, arabinase, galactanase, xylanase, oxidase, a laccase, and/or peroxidase.

12. The detergent composition of claim 10 in the form of a bar, a homogenous tablet, a tablet having two or more layers, a pouch having one or more compartments, a regular or compact powder, a granule, a paste, a gel, or a regular, compact or concentrated liquid.

13. The detergent composition of claim 10, wherein the polypeptide is derived from Exiguobacterium sp AT1b.

14. The detergent composition of claim 10, wherein the polypeptide is derived from Exiguobacterium sibiricum.

15. A method for removing a stain from a surface comprising contacting the surface with the detergent composition of claim 10.
